(19) United States
(12) Patent Application Publication
(10) Pub. No.: US 2024/0273235 A1
(43) Pub. Date: Aug. 15, 2024

(54) DATA ACCESS CONTROL

(71) Applicant: Chen Tech, LLC, Miami Gardens, FL (US)

(72) Inventors: Jen-Ling James Chen, Fort Lauderdale, FL (US); Hernando Celada, Southwest Ranches, FL (US)

(21) Appl. No.: 18/307,145

(22) Filed: Apr. 26, 2023

Related U.S. Application Data
(60) Provisional application No. 63/445,125, filed on Feb. 13, 2023.

(51) Int. Cl. G06F 21/62; G16H 40/20

(52) U.S. Cl. CPC G06F 21/6245; G16H 40/20; G06F 2221/2113; G06F 2221/2141

(57) ABSTRACT

Methods, systems, and apparatus, including computer programs encoded on computer storage media, for controlling data access. One of the methods includes receiving, from a user device associated with an account, a request for data; accessing data from one or more data sources that includes, for each of a plurality of data types, corresponding data values; selecting, from the plurality of data types and using permissions data for the account, one or more data types that the account has permission to access; and providing, to the user device and for each of the one or more data types that the account has permission to access, the corresponding data values.

### CROSS-REFERENCE TO RELATED APPLICATION

This application claims the benefit of U.S. Provisional Application No. 63/445,125, filed on Feb. 13, 2023, the contents of which are incorporated by reference herein.

### BACKGROUND

Databases can include permissions that indicate who has access to a record in the database. The permissions can indicate read permissions, write permissions, or both.

Systems can access data from multiple sources, including multiple databases. When data from each of the sources is from a different provider, and each provider has its own proprietary user interface, the system can use the separate user interfaces to present corresponding data from the corresponding data source.

### SUMMARY

In general, one aspect of the subject matter described in this specification can be embodied in methods that include the actions of receiving, from a user device associated with an account, a request for data; accessing data from one or more data sources that includes, for each of a plurality of data types, corresponding data values; selecting, from the plurality of data types and using permissions data for the account, one or more data types that the account has permission to access; and providing, to the user device and for each of the one or more data types that the account has permission to access, the corresponding data values.

In general, one aspect of the subject matter described in this specification can be embodied in a system including one or more computers and one or more storage devices on which are stored instructions that are operable, when executed by the one or more computers, to cause the one or more computers to perform operations including: receiving, from two or more data sources and using an identifier for a patient, data that indicates two or more characteristics of the patient. The two or more data sources can include lifestyle data, insurance data, and patient data. The operations can further include: accessing a data structure a) representing one or more actions to be performed to improve a health of a patient, relative to a health of the patient prior to performance of the one or more actions b) that was generated using at least some of the data and an action model created from at least portions of the lifestyle data and the patient data from the two or more data sources; and causing, using the data and the data structure, presentation of a user interface that includes i) one or more of the two or more characteristics and ii) second data indicating the one or more actions to be performed to improve the health of the patient, relative to the health of the patient prior to performance of the one or more actions.

Other embodiments of this aspect include corresponding computer systems, apparatus, computer program products, and computer programs recorded on one or more computer storage devices, each configured to perform the actions of the methods. A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by the data processing apparatus, cause the apparatus to perform the actions.

The foregoing and other embodiments can each optionally include one or more of the following features, alone or in combination.

In some implementations, selecting the one or more data types includes selecting, from the plurality of data types and using a role for the account that indicates the permissions data, the one or more data types that the role has permission to access; and providing the corresponding data values includes providing, to the user device and for each of the one or more data types that the role has permission to access, the corresponding data values.

In some implementations, receiving the request for the data includes receiving, from the user device associated the an account, the request for the data for a particular person; accessing the data includes retrieving, from each of the one or more data sources, the data a) for the particular person b) that includes, for each of the plurality of data types, the corresponding data values; and providing the corresponding data values includes providing, to the user device and for each of the one or more data types that the account has permission to access, the corresponding data values for the particular person.

In some implementations, the actions include: in response to receiving the request for data, determining to provide a particular data type to which the account has permission to access; and generating, using data values for one or more of the plurality of data types to which the account does not have permission to access, a value for the particular data type. Providing the corresponding data values can include providing the corresponding data values a) for each of the one or more data types that the account has permission to access b) that include the value for the particular data type.

In some implementations, the actions include: in response to receiving the request for data, determining that data responsive to the request should be presented in a particular user interface; generating instructions for presentation of the particular user interface with one or more fields for the one or more data types that the account has permission to access. Providing the corresponding data values can include providing, to the user device, the instructions and the corresponding data values. The actions can further include: receiving, from a second user device associated with a second, different account, a second request for second data; in response to receiving the second request for second data, determining that data responsive to the second request should be presented in the particular user interface; selecting, from the plurality of data types and using second permissions data for the second account, one or more second data types that the second account has permission to access. The one or more second data types can include at least one data type not included in the one or more data types. The action can further include providing, to the second user device and for each of the one or more second data types that the second account has permission to access, corresponding second data values for each of one or more second data types and second instructions for presentation of the particular user interface with one or more second fields for the one or more second data types that the second account has permission to access.

In some implementations, the account is for a health care employee and the plurality of data types comprise at least one type of medical data.

In some implementations, accessing the data structure includes retrieving, from memory included in the system, the data structure that represents the one or more actions to be performed to improve a health of the patient, relative to a health of the patient prior to performance of the one or more actions.

In some implementations, accessing the data structure includes receiving, from another system, the data structure that represents the one or more actions to be performed to improve a health of the patient, relative to a health of the patient prior to performance of the one or more actions.

In some implementations, accessing the data structure includes generating, using at least some of the data and the action model created from at least portions of the lifestyle data and the patient data from the two or more data sources, the data structure that represents the one or more actions to be performed to improve a health of the patient, relative to a health of the patient prior to performance of the one or more actions.

In some implementations, causing presentation of the user interface includes sending, to a client device and using the data and the data structure, instructions for presentation of the user interface that includes i) the one or more of the two or more characteristics and ii) the second data indicating the one or more actions to be performed to improve the health of the patient, relative to the health of the patient prior to performance of the one or more actions.

In some implementations, causing presentation of the user interface includes presenting, on a display and using the data and the data structure, the user interface that includes i) the one or more of the two or more characteristics and ii) the second data indicating the one or more actions to be performed to improve the health of the patient, relative to the health of the patient prior to performance of the one or more actions.

In some implementations, the operations include, for each of two or more patients including the patient: receiving risk scores that (i) were generated using, as input to a risk model trained using at least portions of the lifestyle data and the patient data from the two or more data sources, characteristics data for the respective patient and (ii) represent a likelihood of a hospital encounter during a time period; ranking, using the risk scores, the two or more patients; selecting, using a threshold value and the ranking of the two or more patients, a plurality of highest risk patients from the two or more patients that have higher likelihoods of hospital encounters than the other patients from the two or more patients; causing presentation of a second user interface that includes, for each of one or more of the plurality of highest risk patients that includes the patient, at least some of the corresponding characteristics data. The user interface and the second user interface can both be interfaces for a single application. The operations can further include receiving input data indicating selection of a user interface element, included in the second user interface, for the patient. Causing, using the data and the data structure, presentation of the user interface that includes i) the one or more of the two or more characteristics and ii) the second data indicating the one or more actions to be performed to improve the health of the patient, relative to the health of the patient prior to performance of the one or more actions can be responsive to receiving the input data indicating selection of the user interface element, included in the second user interface, for the patient.

In some implementations, the operations can include causing, for the patient included in the plurality of highest risk patients and using the risk score for the patient, presentation of a recommendation about at least one of the one or more actions at a higher frequency than if the patient was not included in the plurality of highest risk patients.

In some implementations, causing presentation of the second user interface can include causing presentation of the second user interface that includes, for each of the one or more of the plurality of highest risk patients that includes the patient, (a) at least some of the corresponding characteristics data and (b) a user interface element that initiates removal of a corresponding patient from the plurality of highest risk patients. The operations can include: receiving input data that indicates selection of one of the user interface elements that initiates removal of a second patient from the plurality of highest risk patients; in response to receiving the input data, causing presentation of a third user interface that enables entry of a reason for removal of the second patient from the plurality of highest risk patients or of an option to cancel the removal of the second patient from the plurality of highest risk patients; and selectively maintaining or removing the second patient from the plurality of highest risk patients in response to entry of the reason for removal or the option to cancel the removal.

In some implementations, the operations can include accessing, for a second patient not included in the plurality of highest risk patients, a second risk score that (i) was generating using, as input to the risk model trained using at least portions of the lifestyle data and the patient data from the two or more data sources, second characteristics data for the second patient and (ii) represents a likelihood of a hospital encounter during a time period. Causing presentation of the second user interface can include causing presentation of the second user interface with (a) a first region that includes the second risk score, at least some of the second characteristics data for the second patient, and a recommendation to add the second patient to the plurality of highest risk patients, and (b) a second region that includes, for each of the one or more of the plurality of highest risk patients, at least some of the corresponding characteristics data.

In some implementations, the operations can include: receiving, in response to causing presentation of the second user interface with the first region that includes the recommendation to add the second patient to the plurality of highest risk patients, input data that indicates user selection to add the second patient to the plurality of highest risk patients; and in response to receiving the input data that indicates user selection to add the second patient to the plurality of highest risk patients, causing presentation of an updated second user interface that includes, for each of the one or more of the plurality of highest risk patients including the second patient, at least some of the corresponding characteristics data.

In some implementations, causing presentation of the user interface can include causing, using the data and the data structure, presentation of the user interface that includes i) one or more of the two or more characteristics and ii) second data indicating best practice alerts that indicate the one or more actions to be performed to improve the health of the patient, relative to the health of the patient prior to performance of the one or more actions.

In some implementations, the operations can include: determining, for an account for which the user interface is presented, to provide a particular data type to which the account has permission to access; and generating, using data values for one or more of a plurality of data types to which the account does not have permission to access, a value for the particular data type. Causing presentation of the user interface can include causing presentation of the user interface that includes data of the particular data type to which the account has permission to access including the value for the particular data type that was generated using one or more of the plurality of data types to which the account does not have permission to access.

In some implementations, causing presentation of the user interface can include: retrieving, from the data structure and using a first role associated with a first user device, one or more first characteristics of the patient and the second data indicating a first action to be performed to improve the health of the patient, relative to a heath of the patient prior to performance of the first action; and causing presentation of the user interface i) on the first user device ii) that includes the one or more first characteristics and the second data indicating the first action. The operations can further include: retrieving, from the data structure and using a second, different role associated with a second user device one or more second characteristics of the patient and third data indicating a second, different action to be performed to improve the health of the patient, relative to a heath of the patient prior to performance of the second, different action; and causing presentation of a second user interface i) on the second user device ii) that includes the one or more second characteristics of the patient and the third data indicating the second, different action.

This specification uses the term “configured to” in connection with systems, apparatus, and computer program components. That a system of one or more computers is configured to perform particular operations or actions means that the system has installed on it software, firmware, hardware, or a combination of them that in operation cause the system to perform those operations or actions. That one or more computer programs is configured to perform particular operations or actions means that the one or more programs include instructions that, when executed by data processing apparatus, cause the apparatus to perform those operations or actions. That special-purpose logic circuitry is configured to perform particular operations or actions means that the circuitry has electronic logic that performs those operations or actions.

The subject matter described in this specification can be implemented in various embodiments and may result in one or more of the following advantages. In some implementations, presentation of a unified user interface can reduce a quantity of computer resources used, improve user experience, or both, e.g., compared to systems that have separate applications and present information asynchronously, retrieve data asynchronously, or both. For instance, asynchronous receipt of the data request results can be difficult to view and use in other systems. Due to the unified user interface, the experience of the user may be improved by not having to launch or navigate between multiple user interfaces or applications. In addition, the user interface having interactive elements can reduce the amount of unwanted information in the user interface. The interactive element can conveniently provide the additional information in response to user input. This format can save time for the user, reduce eyestrain for the user, or a combination of these. In some examples, computer resources can be saved by not having to launch multiple applications, accessing information that is likely superfluous, or both. Some examples of reduced computer resources can include network bandwidth, computational cycles, memory, e.g., graphics memory, or a combination of two or more of these.

In some implementations, by providing information and action recommendations in a unified format for multiple users, the chance of the action recommendations being realized can be increased. This can lead to improved health of patients, lower risk of hospitalizations of patients, lower risk of patient re-admittance, or a combination of these. The unified user interface can also increase the chance that the actions taken by users will be properly recorded and stored in the database management system.

In some implementations, the security of data associated with patients can be enhanced, fewer resources can be used to check for permissions before accessing data associated with a patient, or a combination of both. By establishing permissions granted to certain accounts at the data type level instead of the data level, fewer individual records need to be labelled with permissions information, leaving less room for error. Users can spend less time labelling data within a certain data type associated with a patient. Permission being granted at the data type level instead of the data level also demands less computational resources to store and access the permission data. Permission for certain data types can also be allowed globally to particular types of roles in the database management system.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 depicts an example environment in which a database management system controls access to, process, or both, data.

FIG. 2 is an example of a unified user interface for an account.

FIG. 3 is an example of a unified user interface.

FIG. 4 is an example of a unified user interface.

FIG. 5 is a flowchart describing a process that can be carried out by a database management system.

FIG. 6 is a block diagram of a computing system that can be used in connection with computer-implemented methods described in this specification.

Like reference numbers and designations in the various drawings indicate like elements.

### DETAILED DESCRIPTION

Some systems access data retrieved from different data sources. The data from any particular data source can have a corresponding type, which type can be different from the types of data provided by other data sources.

A database management system can combine data from the different data sources to create records, e.g., patient records. Each record can include data from multiple of the different data sources, which data has corresponding data types, and can be for a patient.

User devices can request access to the data. Given security, privacy, or both, concerns, the database management system can limit the types of data to which a requesting user device has access. For instance, although a record for a patient might include five different data types, the database management system can determine permissions for an account associated with the user device, e.g., the account for a user of the user device, and use the permissions to determine a subset of the five different data types to which the account has access. The database management system can then provide, to the user device, data values for the corresponding data types in the subset.

To determine the permissions, the database management system can determine a role for the account. For instance, the database management system can maintain data that indicates roles for each account. Permissions data can indicate the corresponding data types to which each of the roles has access. The database management system can use a role for the account to determine the corresponding permissions data and the data types to which the account has access.

In prior systems, when a user device required access to data from multiple different sources, the user device would present different user interfaces for the multiple different sources. For example, when the user device required insurance data, lifestyle data, and medical service data for a patient, the user device would need to present three different user interfaces, one for each of the different types of data. The user device could use separate applications to present each of these user interfaces, causing the user device to consume more resources than necessary; delay presentation of at least some of the data, e.g., when the user device needs to switch between different user interfaces to display requested data; make it difficult to navigate between all of the data for a single patient; or a combination of two or more of these.

To improve the user interface experience, the database management system can cause presentation of data from different data sources in a single user interface. This enables a user device to display the data more quickly because it does not require the user device to launch multiple applications, each of which presents different data types; does not require the user device to switch between different user interface windows to present data for a single patient; does not require the user device to consume computer resources for presentation of multiple user interfaces; enables presentation of scenario specific user interfaces that present more accurate information, guide to a better decision, or both; or a combination of two or more of these.

The database management system can present, with the data from the different data sources, actionable recommendations that indicate one or more actions that can be performed to improve the health of a patient. These actions can be any appropriate type of action that can reduce a likelihood that the patient will be readmitted, can increase a likelihood that the patient's health will improve, increase the likelihood of rendering more cost-effective care, or a combination of two or more of these. By presenting the actionable recommendations in the same user interface as the data from the different data sources, the database management system can improve a likelihood that at least some of the one or more actions will be performed, in addition to optionally including one or more of the other advantages of providing data in a single user interface.

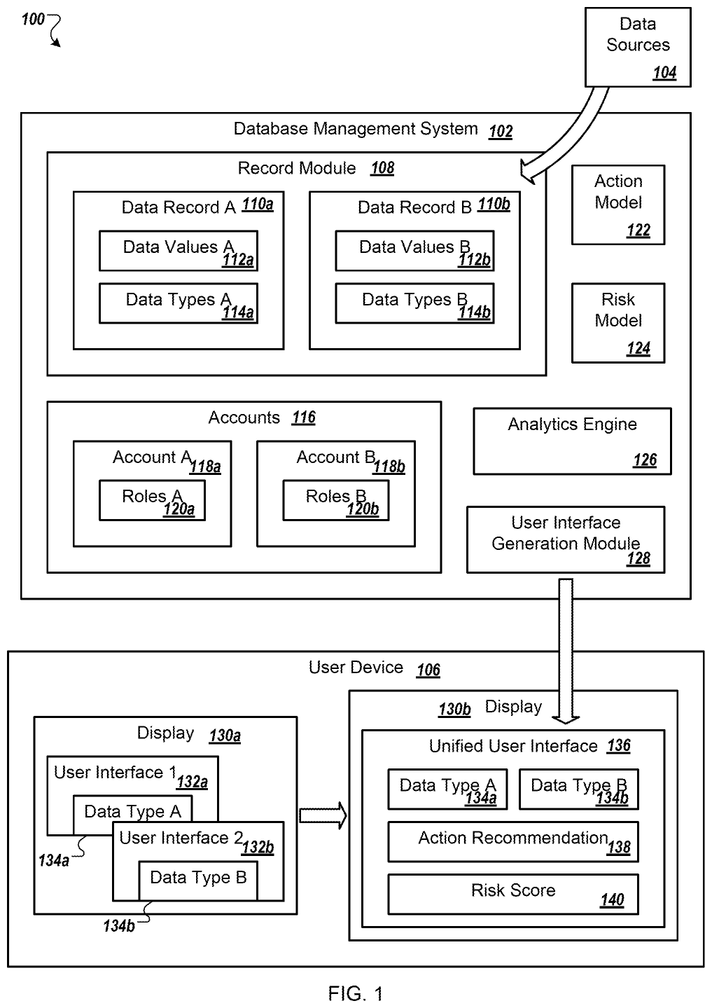

FIG. 1 depicts an example environment 100 in which a database management system 102 controls access to, processes, or both, data. As described in more detail below, the database management system 102 can control access to data using types of the data, e.g., instead of or in addition to particular records or data. The database management system 102 can process data from various data sources 104 and provide a result of the processing to a user device 106. For instance, when the user device 106 has permission to access certain types of data, the database management system 102 can provide values for the certain types of data to the user device 106, optionally along with one or more recommended actions, a risk score, or both, for a patient.

The database management system 102 can receive data of various types from multiple data sources 104, e.g., two or more data sources. The data sources can be any appropriate type of data sources, such as databases, servers, the Centers for Medicare & Medicaid Services (CMS) data, government libraries, payor files, patient records, physician input, remote patient monitoring devices, or a combination of two or more of these, which are external to the database management system 102. The multiple data sources 104 can include medical data sources, non-medical data sources, or a combination of both.

The database management system 102 can receive, from each of the multiple data sources 104, different types of data. For instance, the database management system 102 can receive data from a first data source which data has a first type and the database management system 102 can receive data from a second, different data source, which data has either a second, different type or a third, different type.

The database management system 102 can aggregate data from the multiple data sources 104 into records. The database management system 102 can store the records in a record module 108, e.g., a health record module, which records include aggregate data. For instance, the database management system 102 can receive data from the multiple data sources 104.

The database management system 102 can create one or more records from the data. For instance, the database management system 102 can determine a key for a record. The key can be an identifier for a patient or any other appropriate type of identifier. In some implementations, the key can be an identifier for another individual, such as an employee, clinical provider, or payor. The database management system 102 can use the key to parse the data from the multiple data sources 104 to determine data from at least some of the multiple data sources 104 that corresponds to, e.g., includes, the key. The database management system 102, e.g., a record generation module, can create a record for the key which record includes the determined data that corresponds to the key. For example, the database management system 102 can create data records A-B 110a-b by parsing the data from the data sources 104 when each of the data records A-B 110a-b has a different key, e.g., and is for a different patient.

Each of the data records A-B 110a-b has data values A-B 112a-b. The data values A-B 112a-b have corresponding data types A-B 114a-b. At least some of the data types A-B 114a-b can be the same. In some examples, all of the data types A-B 114a-b for the data records A-B 110a-b are the same. In some examples, only a subset of the data types A-B 114a-b for the data records A-B 110a-b are the same. Sometimes, not all of the data types A-B 114a-b are the same when some of the data sources 104 have data for one key but not another, have data for some data types for a particular key and not all data types, or a combination of both, e.g., in systems with more than two data records.

The database management system 102 can receive a request for data from the user device 106. The request can include a key, or an identifier for a key. Some examples of an identifier for a key, when the data records A-B 110a-b are for patients, are a patient name, or a patient identifier, e.g., that is different than the key.

The database management system 102 can determines an account 118 for the request from multiple accounts 116. For instance, user device 106 can be for the account, e.g., be used by a user who uses the account to log into the user device 106, the database management system 102, or both. The database management system 102 can determine the account using an identifier for the account included in the request, an indication in the request that the request came from the user device 106 to which the account is associated, or using some other appropriate process.

The database management system 102 can determine one or more data types 114 to which the account 118 has access. For example, the multiple accounts 116 can include two or more accounts A-B 118a-b. Each of the two or more accounts A-B 118a-b can have one or more roles A-B 120a-b. For instance, a first account A 118a can have a role A 120a of database administrator, primary care physician, medical assistant, operational staff, referral coordinator, or care administrator. A second account B 118b can have one or more roles B 120b, such as any appropriate combination of network security manager, database administrator, receptionist, or nurse.

The database management system 102 can use the roles A-B 120a-b assigned to an account A-B 118a-b to determine the one or more data types 114 to which the account has access. For example, the database management system 102 can determine that access permissions indicate that accounts assigned to the role A 120a have access to a first and a second data type and accounts assigned to a role B 120b have access to the second and a third data type.

Using the determined data types 114 to which the account has access and the request, the database management system 102 can retrieve data responsive to the request. For instance, the database management system 102 can use the key included in the request to select a record in a database included in the record module 108. The database management system 102 can retrieve, from the selected record, the data values that have the data types 114 to which the account has access. For instance, when the account A 118a has access to a first and a second data type, the database management system 102 can retrieve, from the selected record, e.g., the data record A 110a, the values in the selected data record that have the first and the second data type.

In some implementations, the request received from the user device 106 does not identify any particular record. For instance, the request can have a request type that indicates that the database management system 102 should retrieve data from records for a number of patients. Some examples of request types can include a request for data about the patients who are likely to visit a care facility on the date of the request, a request for data about the patients who are likely to visit the care facility during a time period, or a request for data about high-risk patients.

When the database management system 102 receives a request that does not identify any particular record, the database management system 102 determines the records from which data should be retrieved. The database management system 102 can analyze the request to determine a request type and use the request type to determine the records from which to retrieve data.

When the database management system 102 retrieves data from the data records 110 in the record module 108, similar to the example in which the request identifies a data record, the database management system 102 uses the account 118 for the user device 106 to determine the data types 114 to retrieve. The database management system 102 can then retrieve data values from the data records 110 that have the determined data types 114.

The database management system 102 can provide the retrieved data values to the user device 106. For instance, the database management system 102 can generate instructions for presentation of a user interface and provide the instructions with the retrieved data values to the user device 106. The provision of the instructions with the retrieved data values to the user device 106 can cause the user device 106 to display a user interface that presents the retrieved data values.

In some implementations, the database management system 102 can provide one or more action recommendations, one or more risk scores, or both, with the retrieved data. The database management system 102 can include an action model 122, a risk model 124, or both, from memory that an analytics engine 126 can use to generate the action recommendations and the risk scores, respective.

To generate an action recommendation 138, the analytics engine 126 can access data from the record module 108. For instance, the analytics engine 126 can access a record for a particular patient to generate one or more action recommendations for the particular patient by applying the action model 122 to the parsed values of the fields, which can include at least some of the lifestyle data, demographic data, address data, behavioral data, condition/disease data, insurance data, and patient data received from the various data sources 104, e.g., using the identifier for the patient. The action recommendation 138 can take the form of a data structure representing one or more actions to be performed to improve a health of the patient, relative to a health of the patient prior to performance of the one more actions. The health of a patient prior to performance of the one or more actions is represented by data within the data sources 104, which can include lifestyle data, insurance data, and patient data.

A user interface generation module 128 can generate instructions for presentation of the one or more action recommendations, the one or more risk scores, or both. The instructions can cause the user device 106 to present the characteristics indicated in the request for data and the action recommendation 138, e.g., indicating the one or more actions to be performed to improve the health of the patient, relative to the health of the patient prior to performance of the one or more actions, on a user interface.

In prior systems, a first display 130a would include multiple user interfaces 1-2 132a-b, each of which present different data types A and B 134a-b. Because of the use of multiple user interfaces, e.g., each for a respective application or respective tab in a single application, the user device 106 would need to send multiple requests for corresponding data, each request of which would likely be responsive to separate requests from a user. For instance, the user device 106 might receive a first request for the data type A 134a, which request includes an identifier for a patient, and then the user device might receive a second request for the data type B 134b, which second request also includes the identifier for the patient. The user device 106 would then have to send two requests to the database management system 102, or multiple other systems, and receive corresponding responses, increasing network bandwidth usage.

Instead of requiring the multiple user interfaces 1-2 132a-b, the database management system 102 can receive a single request, e.g., from the user device 106, for the data types A-B 134a-b. The database management system 102 can then generate a response to the request using one or more components, including the user interface generation module 128. The user interface generation module 128 can determine that the data types A and B 134a-b responsive to the request should be presented in a particular user interface. Accordingly, instead of generating instructions for multiple user interfaces or multiple tabs on the first display 130a, the user interface generation module 128 can generate instructions for a single unified user interface 136 for presentation on display 130b, which can include all the data in the first display 130a. For example, the display 130b does not include several windows or applications between which the user needs to navigate to view the data types A-B 134a-b. Data types A-B 134a-b can be presented in separate fields in the unified user interface 136.

The single request can be any appropriate type of request. For instance, the single request can be a login request received from the user device 106. The login request can be a request to login to the application that generates the single unified user interface 136. The login request can be a request to login to the user device 106.

The database management system 102 can generate the response using data from the record module 108, one or more other appropriate sources, or a combination of both. For instance, the database management system 102 can pull data from the other sources according to a schedule and store the data in the record module 108, e.g., that implements a database. In response to receiving the request, the database management system 102 can access data in the record module 108 that is responsive to the request. In some examples, in response to receiving the request, the database management system 102 can query the one or more other appropriate sources for data responsive to the request.

The database management system 102 can determine from where to get responsive data using a type of the data. For instance, the database management system 102 can determine that the data type A 114a can have a first generation date that need not satisfy a time freshness threshold while the data type B 114b can have a second generation data that should satisfy the time freshness threshold. As a result, the database management system 102 can use the data values A 112a in the data record A 110a of the record module 108 while retrieving the data values B 112b from another data source when the second generation data does not satisfy the time freshness threshold.

The unified user interface 136 can be generated such that the information is presented in a way that reduces eye strain for the user, reduces computer resource usage, or both. For example, not all information is presented at once, but rather on demand. When the user hovers a computer mouse over a particular box, more details about what that box means or how the information was generated can be provided in an additional user interface.

In addition to presenting the multiple data types 134, the unified user interface 136 can provide an action recommendation 138 generated by the analytics engine 126. The unified user interface 136 depicts multiple types of data in a convenient way and provides an action recommendation 138 that can help the patient, the user, e. g., a primary care physician or nurse, or both, meet the goal of improving the patient's health by taking action.

The action recommendation 138 can include information about the one or more actions generated as output of the action model 122, as discussed above. For instance, the analytics engine 126 can use the action model to analyze patterns relating to patient behaviors, such as emergency room visits, hospitalizations, rates of contracting diseases, other statistics present in the record module 108, or a combination of these. Examples of action recommendations 138 can include providing a vaccine, changing a prescription, contacting the family of a patient to discuss a care plan, administering a diagnostic exam, scheduling another appointment, or a combination of two or more of these. In some examples, the unified user interface 136 can depict more than one action recommendation 138. After a user interacts with the action recommendation 138, the database management system 102 can receive, from the user device 106, data that represents the user interaction. The record module 108 can use the data to determine updated medical data and send the updated medical data associated with the particular key, e. g., a patient.

The analytics engine 126 can generate a risk score 140 for a particular patient using the risk model 124. The risk score 140 can be any appropriate value, such as a number between 0 and 1, or a percentage. Patients who are more at risk for certain health issues, such as falling or requiring dialysis, can be identified with a value ranging from 0-100, where higher numbers mean higher risk. In some examples, a lower number can indicate higher risk. The analytics engine 126 can determine the risk score 140 using data from the record module 108 and output of the action model 122, the risk model 124, or both. In some examples, the output from the risk model 124 is the risk score 140. Patients with risk scores 140 that indicate the patients are at higher risk may be given priority to earlier appointment dates and encouraged to see their primary care physician more often.

In some examples, the user interface generation module 128 can use the risk score 140 to determine how to generate instructions for the unified user interface 136. For instance, different users associated with accounts with roles A-B 120a-b can use the risk score 140 to inform how the user approaches a particular patient. The user interface generation module 128 can use the risk scores 140 to present information about patients who have a higher risk more prominently in the unified user interface 136 compared to patients who have a lower risk.

The action recommendation 138 and the risk score 140 can be displayed on unified user interfaces 136 for multiple accounts 116, depending on the roles 120 of the accounts 116. For example, both a receptionist who greets the patient and the primary care physician of a patient can be alerted that the patient has not received an annual vaccine. The action recommendation 138 can be tailored to the role of a particular user, account 116, or both. For example, a nurse may be recommended to instruct the patient to visit the receptionist to schedule an appointment before leaving, while the receptionist may be recommended to schedule an appointment for the patient. In some examples, a physician may be recommended to perform an ad hoc visit for a patient who doesn't show up to an appointment. In a further example, a physician may be recommended to not proceed with a particular diagnostic test and instead recommended to perform an alternative test. In some implementations, the action recommendation 138 can indicate that a user should not proceed with an action unless the user can provide a justification. By providing an action recommendation 138 to multiple accounts 116, the chance of an action recommendation 138 being realized can be increased.

In some implementations, the record module 108 can include multiple records for a single key, e.g., for a single patient. For instance, the record module 108 can include, for the single key, a record for each of the data sources 104 from which the database management system 102 received data for the key. This can improve data security by maintaining different types of data for the key in different portions of the record module 108, e.g., a database that stores the records.

The database management system 102 is an example of a system implemented as computer programs on one or more computers in one or more locations, in which the systems, components, and techniques described in this specification are implemented. The user device 106 can include a personal computer, a mobile communication device, a wearable health device, such as a “smart watch”, an augmented reality device, a glucose meter, a Bluetooth weight scale, and other devices that can send and receive data over a network. The network (not shown), such as a local area network (“LAN”), wide area network (“WAN”), the Internet, or a combination thereof, connects the database management system 102, the data sources 104, and the user device 106. The database management system 102 can use a single server computer or multiple server computers operating in conjunction with one another, including, for example, a set of remote computers deployed as a cloud computing service.

The database management system 102 can include several different functional components, including a storage device and a computer with a display monitor. The storage device, computer, or a combination of these, can include one or more data processing apparatuses, can be implemented in code, or a combination of both. For instance, each of the storage device and computer can include one or more data processors and instructions that cause the one or more data processors to perform the operations discussed herein.

The various functional components of the database management system 102 can be installed on one or more computers as separate functional components or as different modules of a same functional component. For example, the components storage device and computer of the database management system 102 can be implemented as computer programs installed on one or more computers in one or more locations that are coupled to each through a network. In cloud-based systems for example, these components can be implemented by individual computing nodes of a distributed computing system.

In some implementations, the performance of accounts 116, e. g., a particular primary care physician or nurse, can be monitored. Some of the data types 134 can reflect how often an account 116 meets a goal, such as regularly seeing a high-risk patient, by following action recommendations 138. Accounts 116 can receive a calculated score having to do with their performance, which can be displayed in the unified user interface 136. Consequently, the unified user interface 136 can function as a performance management system.

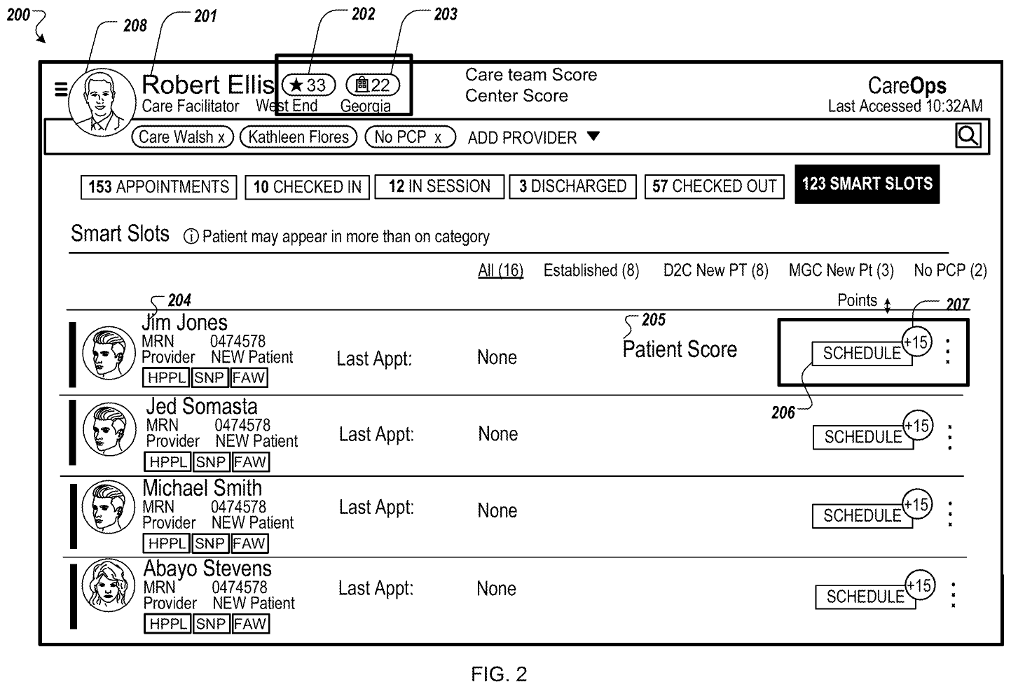

FIG. 2 is an example of a unified user interface 200 for an account. For example, the database management system can customize the unified user interface 200 to display scores for a primary care physician. In some implementations, the unified user interface 200 can display scores other types of roles e.g., a receptionist, a care coordinator, a nurse, or the like. The unified user interface 200 can include an account name field 201, e.g., at the top of the unified user interface 200.

The unified user interface 200 can include one or more scores, such as a care team score 202 and a center score 203. In some implementations, a score can correspond to market-level score, division-level score, region-level score, net promoter score, a percentage of panel seen in a time period, a percentage of a subset of patients seen in a time period, a percentage of patients seen that are in a high-risk group, and slot usage for scheduling. The care team score 202 can be based on data from the record module 108 that concerns measurements of the health of patients that interact with the care team with which the primary care physician is associated. The center score 203 can be based on measurements of the health of patients that visit the center with which the primary care physician is associated. The unified user interface 200 can include a profile picture 208 associated with the primary care physician. The scores can be determined to increase a likelihood that higher risk patients are more likely to be treated before other lower risk patients. The scores can represent which patient should be scheduled when. The scores can represent a ranking of an internal team, e.g., at the center caring for patients. For instance, the care team score 202 can represent how efficiently the employees at a care center schedule patients, a completion rate for patient related tasks, or other appropriate metrics.

For each account, the unified user interface 200 can include statistics such as the number of upcoming appointments, patients who are currently checked in, in session, discharged, checked out, not showing up, and open clinical gaps, other related information, or a combination of two or more of these.

The unified user interface 200 can include a list. The list can be located below the account name field 201 of the primary care physician, the care team score 202, and center score 203. Within the list, the patient name field 204 and risk scores 205 can appear in a row for each patient. In each row can be a schedule button 206 that a user, in this case a primary care physician, can click to schedule the patient. The rows can be arranged according to risk score, e.g., in descending urgency for scheduling the patient, based on the risk scores 205.

FIG. 2 shows a user interface for an account that does not have permission to access the patient score of the listed patients, as one data type. Consequently, a blank appears in the field where the risk score 205 would appear if the account did have permission. When the account has permission to access the patient score, the unified user interface 200 would include the patient score in the list. For instance, as described elsewhere in this specification, the unified user interface 200 can be customized using the data types to which a requesting account has access and present data that corresponds to the data types to which the request account has access. In this way, the same unified user interface 200 can present different data to different accounts, e.g., when the different accounts have different roles.

The unified user interface 200 can include interactive elements that incentivize users to take actions that could improve the health of the patient. In each row in the list of patients can be a schedule button 206 that, when clicked, initiates the process of scheduling an appointment for the patient in that row. In the upper right corner of each schedule button 206 is an icon 207 that indicates how scheduling the patient corresponding to patient name field 204 in that row could increase the care team score 202 of the primary care physician. As a result, the unified user interface 200 presents a “gamified” approach to encourage users to make decisions that positively impact the health of a patient and performance of the user.

For example, the unified user interface 200 can display patients with higher risk scores in a way, e.g., at the top of the unified user interface 200, to encourage a user to take an action regarding the high risk patients, such give them priority in scheduling. For example, the unified user interface 200 can award points for user that schedule more high risk patients. The amount of points awarded can depend on the user and change over time, such as depending on the time of year.

The unified user interface 200 can include features that improve the viewing experience of the user, as well as emphasize patients with high risk scores. For instance, the unified user interface 300 can order the patients according to the risk score of each patient. The user interface generation module can use color, font characteristics, other visual cues, or a combination of two or more of these to quickly provide coded information to the user and draw attention to high-risk patients or other urgent matters. In some examples, by presenting different types of data in the unified user interface 200 that previously was presented in different user interfaces, the unified user interface 200 can improve the viewing experience, reduce computational resource usage, or both.

In some examples, one or more interface components in the unified user interface 200 can be selected. Upon detecting selection of an interface component, a system can cause presentation of more detailed information associated with the interface component. The system can determine the detailed information using a role for an account for which the unified user interface 200 is presented. For instance, an account with a first role, e.g., a receptionist, can have presentation of a first, smaller amount of data while an account with a second role, e.g., a doctor, can have presentation of a second, larger amount of data.

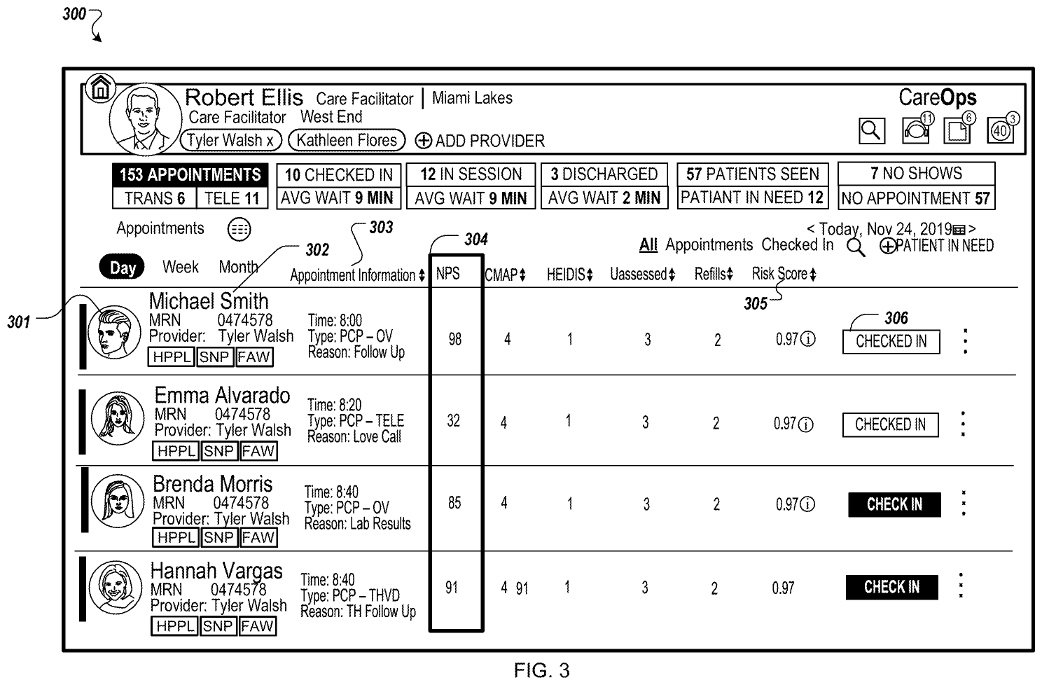

FIG. 3 is an example of a unified user interface 300 associated with a particular account, showing upcoming appointments. User interfaces, such as unified user interface 300, can be customized for a particular primary care physician, a receptionist, or both. In this example, upcoming appointments can be visualized by a series of rows that contain a patient profile picture 301, a patient name field 302, the appointment information field 303, a patient satisfaction score field 304, a risk score field 305, and a status field 306. The unified user interface 300 can be useful for a primary care physician to quickly and efficiently gather information about the day's proceedings, determine which patients to treat first, determine a way in which to treat patients, or a combination of these. Additionally, the value in the patient satisfaction score field 304 can inform how to approach the patient during the appointment. The value in the patient satisfaction score field 304 can be based on daily surveys provided to patients in order to determine how to improve the patient experiences. The knowledge of the value in the patient satisfaction score field 304 can help primary care physicians to prevent disenrollment from disengaged patients, reduced patient health, or both.

In some implementations, the unified user interface 300 can display more information about a patient in a pop-up window in response to a user clicking on an icon representing that patient.

The unified user interface 300 can include features that help manage patients with high risk-scores. Collecting patients with high-risk scores can create a list of high-risk patients. Multiple patients may receive risk scores that were generated using, as input to a risk model trained using at least portions of the lifestyle data and the patient data from the two or more data sources, characteristics data for each patient. The risk scores can represent a likelihood of a hospital encounter during a time period, the risk of the patient getting a specific disease or condition, or the risk of a patient undergoing a negative event, such as a fall. The patients can be ranked by risk scores.

The user interface generation module can cause a user device to present a list of high-risk patients and their corresponding data in the unified user interface 300 as part of a single application, e.g., the unified user interface 136 from FIG. 1. The database management system can determine what patients belong in the high-risk group in multiple ways. For instance, using a threshold value and the ranking of the patients, the database management system can select the patients with risk scores greater than the threshold value. In some implementations, the database management system can initially create the high-risk group by including those patients in the group whose risk scores satisfy, e.g., are greater than or equal to or either, the threshold risk score. The high-risk group can be selectively maintained using the threshold risk score. Patients with risk scores greater than the threshold risk score can automatically remain or be added to the high-risk group.

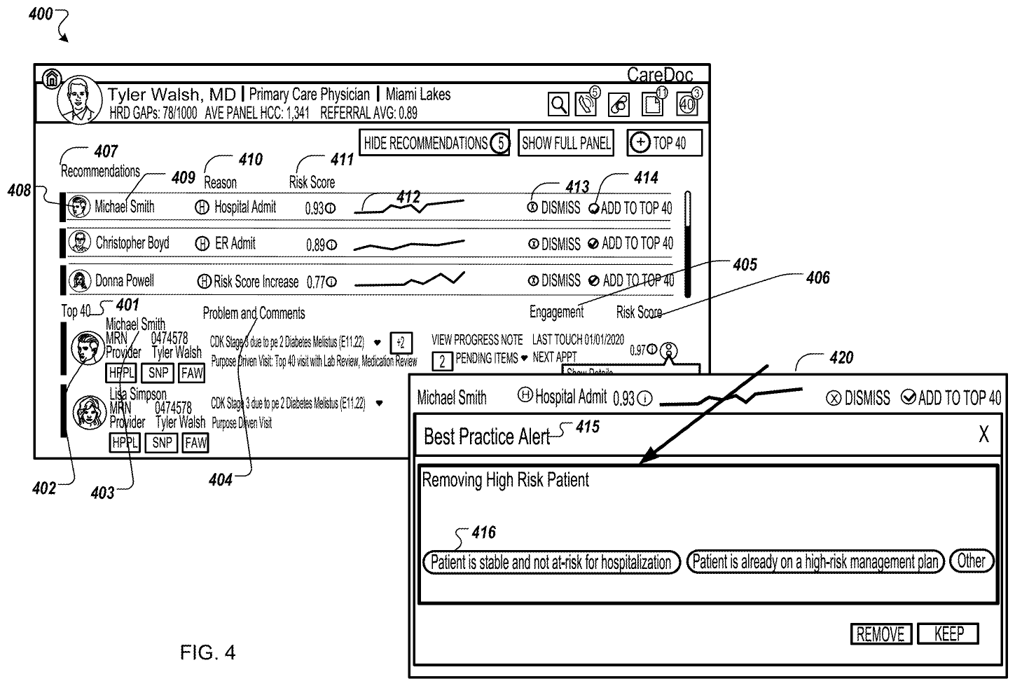

FIG. 4 is an example of a unified user interface 400 associated with a particular account, demonstrating when an additional user interface can appear in response to input data. The high-risk patients can be visualized in a high-risk list 401. In this example, the list of high-risk patients is named “Top 40” 401. In some implementations, the list of patients can be of a different number and based on different criteria. The name of the list can be customized by a database manager or another user associated with a different role. In this example, the name “Top 40” relates the percentage of hospitalizations associated with patients with high-risk scores. The number of patients in the list, the criteria to be included in the list, or both, can be customized.

The unified user interface 400 can include data about each patient in the high-risk list 401. Below the name of the high-risk list 401 can be a series of rows with a patient profile picture 402, the patient name field 403, a field for the list of conditions 404 for the patient, the patient engagement field 405, and the patient risk score field 406.

The unified user interface 400 can include features for adding new patients to the high-risk group. Above the high-risk list 401, recommendations 407 for patients, based on data related to the health of the patients, to be added to the high-risk group can appear. If the size of the high-risk list 401 is fixed, adding a patient can mean automatically removing a patient within the high-risk list 401 with the lowest risk-score, or prompting a user for selection of a patient to remove from the high-risk list 401. If the size of the high-risk list 401 is not fixed, then adding a new patient need not affect the inclusion of the other patients in the high-risk list 401.

The unified user interface 400 can include data about the patients being recommended to the high-risk list 401. The recommendation 407 can include a patient profile picture 408, a field for the patient name 409, a field with the reason 410 that the patient is recommended to be included in the high-risk group, a patient risk score field 411, and a plot 412 allowing the user to visualize how the patient risk score has changed over time. In some implementations, the database management system recommends a patient because the risk-score of the patient has passed a threshold value, which can be visualized by a plot 412 with increasing values toward the right end. Within the recommendation 407 can be two icons that allow a user to respond to the recommendation. Clicking a dismiss icon 413 can dismiss the recommendation 407, keeping a patient out of the high-risk list 401. Clicking an accept icon 414 can add the patient to the high-risk list 401.

The unified user interface 400 can include a user interface element that initiates removal of a corresponding patient from the high-risk list 401. In response to receiving the input data corresponding to initiating removal of a patient from the high-risk group, e. g., clicking a remove icon similar to the dismiss icon 413, the user interface generation module can present another user interface 420 that enables entry of a reason for removal a patient or an option to cancel the removal of the patient from the high-risk patients.

The user interface 420 can include a best practice alert 415 that prompts the user to supply a reason for either dismissing the recommendation 407, e.g., with a keep button, or removing the patient from the high-risk list 401. There can be multiple-choice format icons 416 with answers such as “the patient is stable and not at risk for hospitalization” or “the patient is already on the high-risk management plan.” In some implementations, there can be an option for free form response, an option that indicates something other than the multiple-choice options, or both. Within the user interface 420, after selection of a reason to remove a patient, the unified user interface 400 can enable selection of a remove button that removes the corresponding patient from the high-risk list 401.

The unified user interfaces 400 can be a unified user interface, as it presents information that was previously presented in multiple different user interfaces for different corresponding applications. The user interface can contain a primary user interface and an additional user interface 420, e.g., the user interface 420 can be a popup that partially overlays the unified user interface 400 and disappears after receipt of input indicating whether to cancel the remove or a reason for removal. For the purposes of explanation, the user interface 420 can extend beyond the boundary of unified user interface 400, but it could be contained within the borders of unified user interface 400.

The unified user interface 400 can include elements that allow data input from a user. In some implementations, the unified user interface 400 can include an option enabling a user to manually add a patient to the high-risk list 401, even when the patient is not included in the recommendations 407. Before, after, or at substantially the same time that a user associated with an account performs an action indicating selection to add a patient to the high-risk group, the database management system can generate using, as input to a risk model trained using at least portions of the lifestyle data and the patient data from multiple data sources, characteristics data for the patient that represents a likelihood of a hospital encounter during a time period. In response to receiving the input data that indicates user selection to add the patient to high-risk group, the user interface generation module can cause presentation of an updated user interface that can include the updated high-risk list 401 and the corresponding characteristics of the patients included in that group.

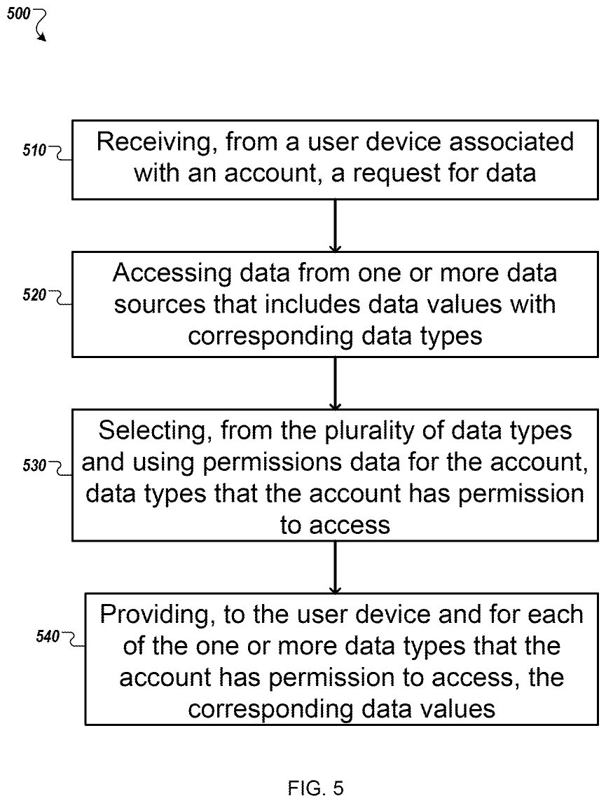

FIG. 5 is a flow diagram of an example of a process 500 for a database management system. Process 500 can be carried out by system of one or more computers and one or more storage devices that store instructions that can be operated by one or more computers. For example, the process 500 can be performed by the database management system 102 from the example environment 100, e.g., to interact with the user device 106.

The database management system can receive a request for data from a user device associated with an account (510). The request can be for any appropriate type of data, such as health data, an action recommendation, a risk score, a machine learning model, or software code. The type of the data can depend on the account and the different types of data to which the account has access. For instance, a primary care physician can have access to health data, an action recommendation, and a risk score while a programmer or a developer might have access to a machine learning model or software code.

The database management system can access data from one or more data sources that includes, for each of the multiple data types, corresponding data types (520). For instance, the data management system can receive, from two or more data sources and using an identifier for a patient, data that indicates two or more characteristics of the patient. The two or more data sources can include lifestyle data, insurance data, and patient data. In some examples, accessing the data can be responsive to receipt of the request for data from the user device. In some implementations, accessing the data can occur prior to receipt of the request for data.

In some implementations, the data sources include payor files, medical records, discharge summary, eligibility information, and social determinates of health (SDOH) results from a survey).

Even though this data can be in different formats and have different default representations, the database management system can collate the data values corresponding to various data types such that it can eventually be presented to the user device in a unified user interface.

Depending on what was included in the request for data, accessing data can include accessing a data structure that represents one or more actions to be performed to improve a health of a patient, relative to a health of the patient prior to performance of the one or more actions. This data structure can be generated using at least some of the data and an action model created from at least portions of the lifestyle data and the patient data from the two or more data sources. For example, the data structure can be an action recommendation. If an action recommendation associated with the request for data has already been generated at the time of the request for data, then the action recommendation can be accessed by retrieving it from memory.

In the data sources, the database management system, or both, a portion of the data can have limitations related to privacy and permissions, e. g., HIPPA requirements. This permissions can be based on account, a role for corresponding accounts, data type, or a combination of two or more of these.

The database management system can select, from the multiple data types and using permissions data for the account, one or more data types that the account has permission to access (530). For example, the account of a primary care physician may only have access to certain data if the patient associated with that data has signed official consent forms.

In some implementations, permissions data can be uniform for each type of role, data type, or a combination of both. For example, a primary care physician could have permission to access any health data associated with a patient that the primary care physician sees, while a receptionist may have access to the insurance data of any patient that visits a particular care facility. In some implementations, each account can have tailored permissions. In some implementations, permissions to access all data can be granted to every account in the database management system.

The database management system can provide data values corresponding to the request for data and allowed by the permissions data associated with the account to the user interface on the user device (540). Sometimes the data values can include a data structure representing actions to be performed to improve a health of a patient.

A user interface generation module within the database management system can present, or cause presentation of, a user interface that includes one or more of the two or more characteristics and additional data indicating the one or more actions to be performed to improve the health of the patient, relative to the health of the patient prior to performance of the one or more actions, e. g., an action recommendation. The actions to improve the health of a patient generated by using the data and an action model can be considered a data type, which a user device associated with a device can request. The display can present data of different types in a unified user interface. All or a portion of the unified user interface can be presented within a display.

In some implementations, the database management system can provide output additional to data values corresponding to the request for data. For example, database management system can receive a request for the vitals of a patient, then provide the vitals of the patient and an unrequested action recommendation.

In some examples, the process 500 can include other steps in addition to providing the user device with a user interface with the data types. In some instances, the database management system can receive data that indicates two more characteristics of patient from the data sources, which can include lifestyle data, insurance data, and patient data.

In certain implementations, when the action recommendation is provided, the database management system can send instructions for presentation of the user interface to a client device. The instructions can include multiple characteristics indicated by the request for data and data indicating the one or more actions to be performed to improve the health of the patient, relative to the health of the patient prior to performance of the one or more actions.

The order of steps in the process 500 described above is illustrative only, and the process leading to providing and presenting data to the user interface on the user device can be performed in different orders. For example, one or more actions to improve the health of a patient may be generated using data and an action model before a user device associated with a device makes a request for data.

The database management system can present a user interface with an action recommendation at a different time from when it provides other data values corresponding to a user request. Alternatively, the action recommendation and other data values corresponding can be presented substantially concurrently.

In some examples, selecting, from multiple data types and using permissions data for the account, data types that the account has permission to access (530) could occur before accessing the data from one or more data sources that include data values with corresponding data types (520).

In some implementations, the process 500 can include additional steps, fewer steps, or some of the steps can be divided into multiple steps. For example, the selecting, from the multiple data types and using permissions data for the account, one or more data types that the account has permission to access (530) can include selecting, from the multiple data types and using a role for the account that indicates the permissions data, the one or more data types that the role has permission to access.

Selecting, from the multiple data types and using permissions data for the account, data types that the account has permission to access (530) can also include providing, to the user device and for each of the one or more data types that the role has permission to access, the corresponding data values.

Receiving a request for data from a user device associated with an account (510) can include a request for the data for a particular person, followed by accessing the data. The data can be accessed by retrieving, from each of the one or more data sources, the data for the particular person, including for each of the multiple data types, the corresponding data values. The database management system can provide, to the user device and for each of the one or more data types that the account has permission to access, the corresponding data values for the particular person.

A user associated with an account may make a request from multiple data types, some of which the user does not have permission to access. In some instances, a value for particular data type that an account does have permission to access is generated from data values corresponding to a data type to which the account does not have access. For example, a receptionist who is responsible for scheduling patients may not have permission to access data related to the health of the patient. The receptionist might, however, have permission to access the risk score of the patient, which can be based on at least a portion of data related to the health of the patient. When s situation like this arises, the database management system can determine to provide a particular data type, e. g., a risk score, to which the account has permission to access, in response to receiving a request for data. Using data values for the one or more data types to which the account does not have permission to access, e. g., health records, the database management system can generate a value for the particular data type. When providing the corresponding data values, the database management system can include providing the corresponding data values for each of the one or more data types that the account has permission to access, which include the value for the particular data type.

Different accounts can vary in what data types they have permission to access. The accessed and provided data also depends on the request for data. For at least these reasons, the process that follows a user associated with an account making a request for data can yield different results depending on the account and request for data. In response to receiving a request for first data from a first account on a first user device, the database management system can determine that data responsive to the request should be presented in a particular user interface. The user interface generation module can generate instructions for presentation of the particular user interface with one or more fields for the one or more data types that the first account has permission to access. The database management system can provide the corresponding data values and instructions to the first user device.

Then a second account, having different permissions from the first account, can make a request for second data from a second user device. In response to the second request for second data, the database management system can determine that the data corresponding to the second request should be presented in the same user interface as for the first user device. The database management system can select one or more second data types that the second account has permission to access from multiple data types, using second permissions data for the second account.

In some instances, one or more second data types include at least one data type not included in the one or more first data types. In some instances, one or more first data types include at least one data type not included in the one or more second data types. Both the first and second data types can include data types not included in the other.

The user interface generation module can provide to the second user device and for each of the one or more second data types that the second account has permission to access, corresponding second data values for each of one or more second data types and second instructions for presentation of the particular user interface with one or more second fields for the one or more second data types that the second account has permission to access.

Though two accounts may have access to different data types, the user interface presenting the data values corresponding to the data types to which each account has permission to access can be the same. For example, a user interface may allow space to present a reason for why a particular patient is in the high-risk group of patients. A primary care physician may have permission to access the data type corresponding to a reason for why particular patient is in the high-risk group of patients and may see a data value in the field in the user interface. A receptionist may not have permission to access the data type, so may see a blank in the field corresponding to a reason for why a particular patient is in the high-risk group of patients.

A similar process could be repeated for more than two user devices associated with different user accounts. Further, a single user account can be associated with multiple user devices, e. g., being able to log in from various, shared computers, or a user device can be associated with multiple user accounts, e. g., multiple nurses use the same computer that remains in the same location.

Personally identifiable information related to patients isn't necessarily included in the record module. HIPPA and patient-provided consent will inform how and when personally identifiable data is stored and supplied to the user interface. In addition, certain data may be anonymized in one or more ways before it is stored or used, so that personally identifiable information is removed. For example, a user's identity may be anonymized so that no personally identifiable information can be determined for the user, or a user's geographic location may be generalized where location information is obtained (such as a city, ZIP code, or state level), so that a particular location of a user cannot be determined. Thus, the user may have control over how information is collected about him or her and used by a database management system.

A number of implementations have been described. Nevertheless, it will be understood that various modifications may be made without departing from the spirit and scope of the disclosure. For example, various forms of the flows shown above may be used, with steps re-ordered, added, or removed.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus” refers to data processing hardware and encompasses all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Computers suitable for the execution of a computer program include, by way of example, general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a smart phone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., LCD (liquid crystal display), OLED (organic light emitting diode) or other monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an Hypertext Markup Language (HTML) page, to a user device, e.g., for purposes of displaying data to and receiving user input from a user interacting with the user device, which acts as a client. Data generated at the user device, e.g., a result of the user interaction, can be received from the user device at the server.

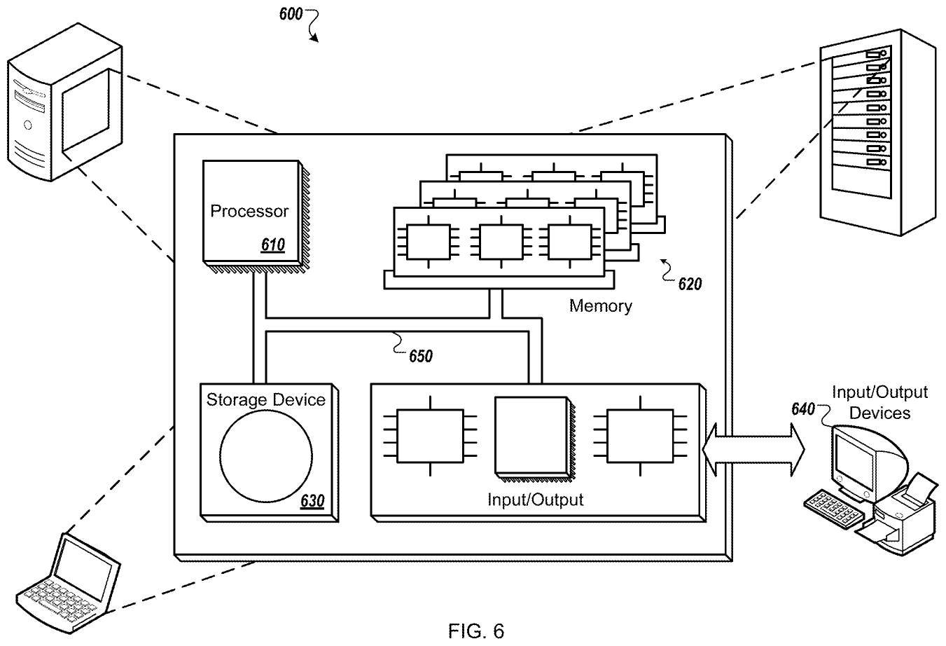

FIG. 6 is a block diagram of a computing system that can be used in connection with computer-implemented methods described in this specification. The system 600 can be used for the operations described in association with any of the computer-implemented methods described previously, according to one implementation. The system 600 includes a processor 610, a memory 620, a storage device 630, and an input/output device 640. Each of the components 610, 620, 630, and 640 are interconnected using a system bus 650. The processor 610 is capable of processing instructions for execution within the system 600. In one implementation, the processor 610 is a single-threaded processor. In another implementation, the processor 610 is a multi-threaded processor. The processor 610 is capable of processing instructions stored in the memory 620 or on the storage device 630 to display graphical information for a user interface on the input/output device 640.

The memory 620 stores information within the system 600. In one implementation, the memory 620 is a computer-readable medium. In one implementation, the memory 620 is a volatile memory unit. In another implementation, the memory 620 is a non-volatile memory unit.

The storage device 630 is capable of providing mass storage for the system 600. In one implementation, the storage device 630 is a computer-readable medium. In various different implementations, the storage device 630 may be a floppy disk device, a hard disk device, an optical disk device, or a tape device.

The input/output device 640 provides input/output operations for the system 600. In one implementation, the input/output device 640 includes a keyboard and/or pointing device. In another implementation, the input/output device 640 includes a display unit for displaying graphical user interfaces.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

In each instance where an HTML file is mentioned, other file types or formats may be substituted. For instance, an HTML file may be replaced by an XML, JSON, plain text, or other types of files. Moreover, where a table or hash table is mentioned, other data structures (such as spreadsheets, relational databases, or structured files) may be used.

Particular embodiments of the invention have been described. Other embodiments are within the scope of the following claims. For example, the steps recited in the claims, described in the specification, or depicted in the figures can be performed in a different order and still achieve desirable results. In some cases, multitasking and parallel processing may be advantageous.

What is claimed is:

1. A computer-implemented method comprising:
receiving, from a user device associated with an account, a request for data;
accessing data from one or more data sources that includes, for each of a plurality of data types, corresponding data values;
selecting, from the plurality of data types and using permissions data for the account, one or more data types that the account has permission to access; and
providing, to the user device and for each of the one or more data types that the account has permission to access, the corresponding data values.

2. The method of claim 1, wherein:
selecting the one or more data types comprises selecting, from the plurality of data types and using a role for the account that indicates the permissions data, the one or more data types that the role has permission to access; and
providing the corresponding data values comprises providing, to the user device and for each of the one or more data types that the role has permission to access, the corresponding data values.

3. The method of claim 1, wherein:
receiving the request for the data comprises receiving, from the user device associated the an account, the request for the data for a particular person;
accessing the data comprises retrieving, from each of the one or more data sources, the data a) for the particular person b) that includes, for each of the plurality of data types, the corresponding data values; and
providing the corresponding data values comprises providing, to the user device and for each of the one or more data types that the account has permission to access, the corresponding data values for the particular person.

4. The method of claim 1, comprising:
in response to receiving the request for data, determining to provide a particular data type to which the account has permission to access; and
generating, using data values for one or more of the plurality of data types to which the account does not have permission to access, a value for the particular data type,
wherein providing the corresponding data values comprises providing the corresponding data values a) for each of the one or more data types that the account has permission to access b) that include the value for the particular data type.

5. The method of claim 1, comprising:
in response to receiving the request for data, determining that data responsive to the request should be presented in a particular user interface;
generating instructions for presentation of the particular user interface with one or more fields for the one or more data types that the account has permission to access, wherein providing the corresponding data values comprises providing, to the user device, the instructions and the corresponding data values;
receiving, from a second user device associated with a second, different account, a second request for second data;
in response to receiving the second request for second data, determining that data responsive to the second request should be presented in the particular user interface;
selecting, from the plurality of data types and using second permissions data for the second account, one or more second data types that the second account has permission to access, wherein the one or more second data types include at least one data type not included in the one or more data types; and
providing, to the second user device and for each of the one or more second data types that the second account has permission to access, corresponding second data values for each of one or more second data types and second instructions for presentation of the particular user interface with one or more second fields for the one or more second data types that the second account has permission to access.

6. The method of claim 1, wherein the account is for a health care employee and the plurality of data types comprise at least one type of medical data.

7. A system comprising one or more computers and one or more storage devices on which are stored instructions that are operable, when executed by the one or more computers, to cause the one or more computers to perform operations comprising:
receiving, from two or more data sources and using an identifier for a patient, data that indicates two or more characteristics of the patient, wherein the two or more data sources include lifestyle data, insurance data, and patient data;
accessing a data structure a) representing one or more actions to be performed to improve a health of a patient, relative to a health of the patient prior to performance of the one or more actions b) that was generated using at least some of the data and an action model created from at least portions of the lifestyle data and the patient data from the two or more data sources; and
causing, using the data and the data structure, presentation of a user interface that includes i) one or more of the two or more characteristics and ii) second data indicating the one or more actions to be performed to improve the health of the patient, relative to the health of the patient prior to performance of the one or more actions.

8. The system of claim 7, wherein accessing the data structure comprises receiving, from another system, the data structure that represents the one or more actions to be performed to improve a health of the patient, relative to a health of the patient prior to performance of the one or more actions.

9. The system of claim 7, wherein accessing the data structure comprises generating, using at least some of the data and the action model created from at least portions of the lifestyle data and the patient data from the two or more data sources, the data structure that represents the one or more actions to be performed to improve a health of the patient, relative to a health of the patient prior to performance of the one or more actions.

10. The system of claim 7, wherein causing presentation of the user interface comprises sending, to a client device and using the data and the data structure, instructions for presentation of the user interface that includes i) the one or more of the two or more characteristics and ii) the second data indicating the one or more actions to be performed to improve the health of the patient, relative to the health of the patient prior to performance of the one or more actions.

11. The system of claim 7, wherein causing presentation of the user interface comprises presenting, on a display and using the data and the data structure, the user interface that includes i) the one or more of the two or more characteristics and ii) the second data indicating the one or more actions to be performed to improve the health of the patient, relative to the health of the patient prior to performance of the one or more actions.

12. The system of claim 7, the operations comprising:
for each of two or more patients including the patient:
receiving risk scores that (i) were generated using, as input to a risk model trained using at least portions of the lifestyle data and the patient data from the two or more data sources, characteristics data for the respective patient and (ii) represent a likelihood of a hospital encounter during a time period;
ranking, using the risk scores, the two or more patients;
selecting, using a threshold value and the ranking of the two or more patients, a plurality of highest risk patients from the two or more patients that have higher likelihoods of hospital encounters than the other patients from the two or more patients;
causing presentation of a second user interface that includes, for each of one or more of the plurality of highest risk patients that includes the patient, at least some of the corresponding characteristics data, wherein the user interface and the second user interface are both interfaces for a single application; and
receiving input data indicating selection of a user interface element, included in the second user interface, for the patient,
wherein causing, using the data and the data structure, presentation of the user interface that includes i) the one or more of the two or more characteristics and ii) the second data indicating the one or more actions to be performed to improve the health of the patient, relative to the health of the patient prior to performance of the one or more actions is responsive to receiving the input data indicating selection of the user interface element, included in the second user interface, for the patient.

13. The system of claim 12, the operations comprising:
causing, for the patient included in the plurality of highest risk patients and using the risk score for the patient, presentation of a recommendation about at least one of the one or more actions at a higher frequency than if the patient was not included in the plurality of highest risk patients.

14. The system of claim 12,
wherein causing presentation of the second user interface comprises causing presentation of the second user interface that includes, for each of the one or more of the plurality of highest risk patients that includes the patient, (a) at least some of the corresponding characteristics data and (b) a user interface element that initiates removal of a corresponding patient from the plurality of highest risk patients,
the operations comprising:
receiving input data that indicates selection of one of the user interface elements that initiates removal of a second patient from the plurality of highest risk patients;
in response to receiving the input data, causing presentation of a third user interface that enables entry of a reason for removal of the second patient from the plurality of highest risk patients or of an option to cancel the removal of the second patient from the plurality of highest risk patients; and
selectively maintaining or removing the second patient from the plurality of highest risk patients in response to entry of the reason for removal or the option to cancel the removal.

15. The system of claim 12, the operations comprising:
accessing, for a second patient not included in the plurality of highest risk patients, a second risk score that (i) was generating using, as input to the risk model trained using at least portions of the lifestyle data and the patient data from the two or more data sources, second characteristics data for the second patient and (ii) represents a likelihood of a hospital encounter during a time period,
wherein causing presentation of the second user interface comprises causing presentation of the second user interface with (a) a first region that includes the second risk score, at least some of the second characteristics data for the second patient, and a recommendation to add the second patient to the plurality of highest risk patients, and (b) a second region that includes, for each of the one or more of the plurality of highest risk patients, at least some of the corresponding characteristics data.

16. The system of claim 15, the operations comprising:
receiving, in response to causing presentation of the second user interface with the first region that includes the recommendation to add the second patient to the plurality of highest risk patients, input data that indicates user selection to add the second patient to the plurality of highest risk patients; and
in response to receiving the input data that indicates user selection to add the second patient to the plurality of highest risk patients, causing presentation of an updated second user interface that includes, for each of the one or more of the plurality of highest risk patients including the second patient, at least some of the corresponding characteristics data.

17. The system of claim 7, wherein causing presentation of the user interface comprises causing, using the data and the data structure, presentation of the user interface that includes i) one or more of the two or more characteristics and ii) second data indicating best practice alerts that indicate the one or more actions to be performed to improve the health of the patient, relative to the health of the patient prior to performance of the one or more actions.

18. The system of claim 7, the operations comprising:
determining, for an account for which the user interface is presented, to provide a particular data type to which the account has permission to access; and
generating, using data values for one or more of a plurality of data types to which the account does not have permission to access, a value for the particular data type,
wherein causing presentation of the user interface comprises causing presentation of the user interface that includes data of the particular data type to which the account has permission to access including the value for the particular data type that was generated using one or more of the plurality of data types to which the account does not have permission to access.

19. The system of claim 7, wherein causing presentation of the user interface comprises:
retrieving, from the data structure and using a first role associated with a first user device, one or more first characteristics of the patient and the second data indicating a first action to be performed to improve the health of the patient, relative to a heath of the patient prior to performance of the first action; and
causing presentation of the user interface i) on the first user device ii) that includes the one or more first characteristics and the second data indicating the first action,
the operations comprising:
retrieving, from the data structure and using a second, different role associated with a second user device one or more second characteristics of the patient and third data indicating a second, different action to be performed to improve the health of the patient, relative to a heath of the patient prior to performance of the second, different action; and
causing presentation of a second user interface i) on the second user device ii) that includes the one or more second characteristics of the patient and the third data indicating the second, different action.

20. A non-transitory computer storage medium encoded with instructions that, when executed by one or more computers, cause the one or more computers to perform operations comprising:
receiving, from a user device associated with an account, a request for data;
accessing data from one or more data sources that includes, for each of a plurality of data types, corresponding data values;
selecting, from the plurality of data types and using permissions data for the account, one or more data types that the account has permission to access; and
providing, to the user device and for each of the one or more data types that the account has permission to access, the corresponding data values.
